(19) United States
(12) Patent Application Publication
(10) Pub. No.: US 2009/0306565 A1
(43) Pub. Date: Dec. 10, 2009

(54) REHABILITATION WALKING BOOT

(76) Inventor: Shu-Chen CHAN, Fongyuan City (TW)

Correspondence Address: EGBERT LAW OFFICES, 412 MAIN STREET, 7TH FLOOR, HOUSTON, TX, 77002, US

(21) Appl. No.: 12/133,932

(22) Filed: Jun. 5, 2008

(51) Int. Cl. A61F 5/00

(52) U.S. Cl. 602/28; 602/27; 602/23

(57) ABSTRACT

The present invention is a rehabilitation walking boot, which includes a foot support and two leg supports. The foot support includes a top surface, a bottom surface and two lateral edges. The two lateral edges are configured with slots for insertion and fixation by the inserts of the two leg supports. The leg support insert is configured with a flexible positioning locker protruded on one side, and one side of the slots of the foot support lateral edges is configured with a locking part. The side walls of the slots are configured with perforated fixing holes. The rehabilitation walking boot can be dismantled more conveniently, and the leg support can use either combined type or fixed type linkages to the foot support.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to an improved orthopedic rehabilitation walking boot, and specifically to an innovative boot which features easier dismantling with convenience and safety. In particular, the leg support can be linked to the foot support in either a combined pattern or immobile pattern.

2. Description of Related Art Including Information Disclosed Under 37 CFR 1.97 and 37 CFR 1.98.

The rehabilitation walking boot described in the present invention is a combination of a foot support and a leg support, helping rehabilitation of acute or severe ankle sprain and patients with injured legs initially fixed via a cast. After cast removal, this kind of rehabilitation walking boot will be an aid for fixation of the bone structures of the foot and leg.

The prior art rehabilitation walking boot is a structure in which the foot support and the leg support are combined into one body. To make it more practicable, the industry developed a new kind of rehabilitation walking boot with a structure in which the foot support and the leg support are linked together in a combined pattern. In known combined-pattern rehabilitation walking boots, the locking structure of the leg support is made up of two flexible claws configured in two directions with spacing. Between the bidirectional claws is locking plate to retain the two flexible claws and to fix the linkage.

However, practical applications of this known structure have found another problem. As soon as the claws are linked together, it is very difficult to release the fixed linkage. To release it, the locking plate must be lifted by force. The two flexible claws are pressed inward, and the leg support is pulled out. The difficulty and inconvenience in dismantling the leg support is obvious, as it requires application of forces simultaneously in several directions.

The rehabilitation walking boot currently available on the market can be divided into two categories: one with a plastic leg support and the other with a metal leg support. The leg support of the combined-pattern rehabilitation walking boot described above is made of plastics. Rehabilitation walking boots with a metal leg support is usually of a fixed pattern. At present, as these two patterns are incompatible, when facing orders for two different types of products, the industry has to make molds for production respectively. This obviously leads to higher costs and is undesirable for the economic benefits of the industry.

Thus, to overcome the aforementioned problems of the prior art, it would be an advancement in the art to provide an improved structure that can significantly improve efficacy.

Therefore, the inventor has provided the present invention of practicability after deliberate design and evaluation based on years of experience in the production, development and design of related products.

### BRIEF SUMMARY OF THE INVENTION

With the unique innovation that said leg support insert is configured with a flexible positioning locker which protrudes on one side, the leg support can be dismantled by only pressing the flexible positioning locker inward and concurrently pulling the leg support out. It is an advancement because dismantling the leg support is more convenient and efficient.

With the unique innovation that the side walls of the slots of the foot support are configured with perforated fixing holes, the leg support and foot support can be fixed using rivets or bolts. Hence, the leg support can link to the foot support in a combined pattern or fixed pattern. The industry can choose a leg support of combined pattern or fixed pattern according to different demands of customers. The foot support is compatible with all and does not need a new mold for production. It is economically beneficial for the industry because manufacturing costs are reduced.

With the structure of the top surface of the foot support configured with multiple partitioned concave spaces and partitions, the material for manufacturing the foot support is conserved. The weight of the foot support is also reduced. While keeping the structural strength, the burden on the user is reduced.

With the structure of the foot support bottom surface configured with a protrusion to fit a sole cover, a combined sole structure is formed. Manufacturing and assembly is more simple and efficient, and it is easier to change the sole.

With the in-between portion of the sole cover landing part and front upswept part of the foot support, there are at least 7 landing sections with different included angles within the horizon. When the landing surface of the sole cover touches the ground, an optimum leg movement reaction can be achieved. And consequently, when walking, the users can move their legs more smoothly, and the possible oppression on the wound can be minimized.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 depicts an assembled perspective view of a preferred embodiment of the present invention.

FIG. 2 depicts an exploded perspective view of a preferred embodiment of the present invention.

FIG. 3 depicts a partially enlarged perspective view of the present invention.

FIG. 4 depicts a sectional view of the combined action of the present invention.

FIG. 5 depicts another sectional view of the combined action of the present invention.

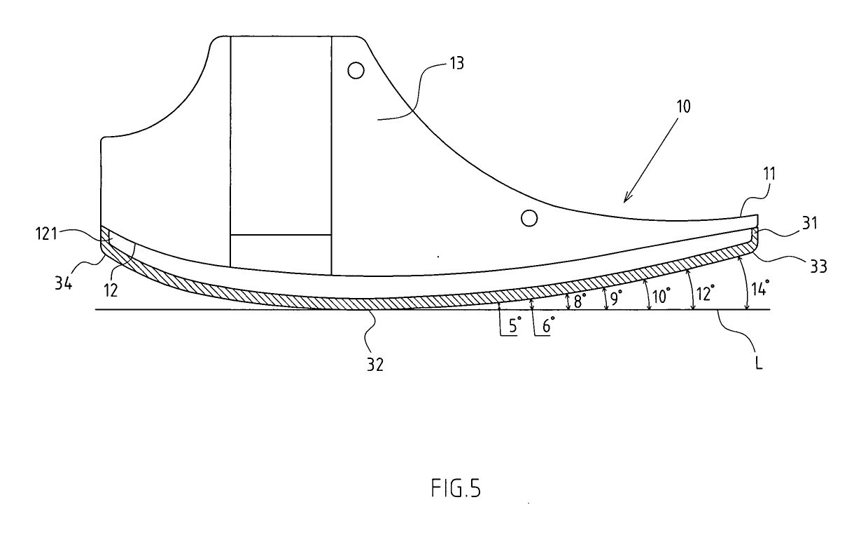

FIG. 6 depicts a sectional view of an embodiment of the present invention with the foot support 1 linking the leg support in a fixed type.

FIG. 7 depicts a an exploded perspective view of the sole cover structure of the foot support of the present invention.

### DETAILED DESCRIPTION OF THE INVENTION

The features and the advantages of the present invention will be more readily understood upon a thoughtful deliberation of the following detailed description of a preferred embodiment of the present invention with reference to the accompanying drawings.

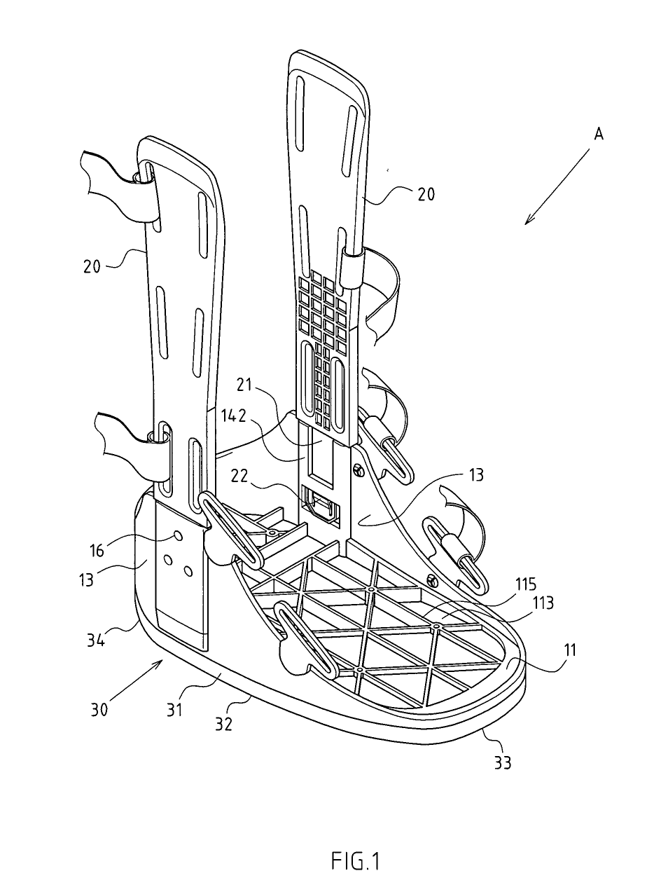

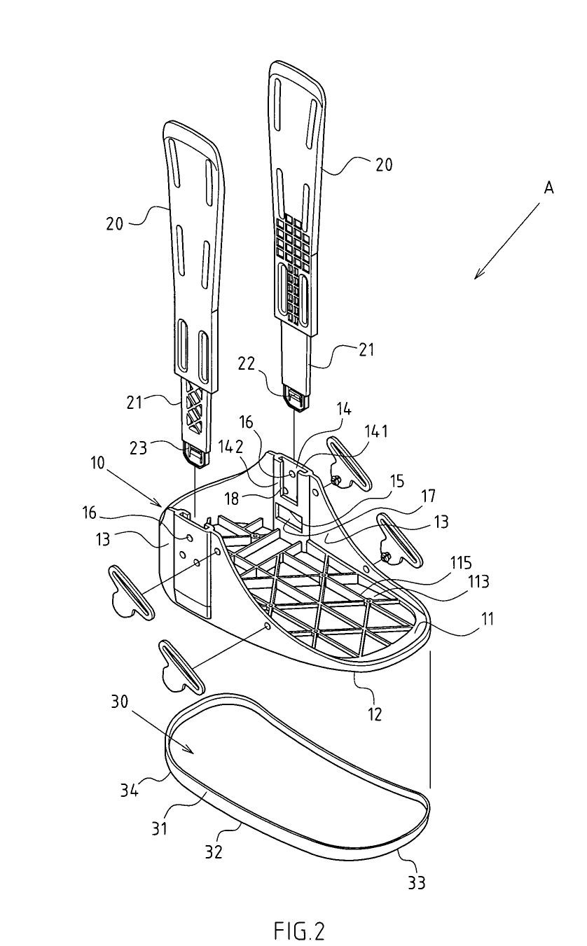

FIGS. 1, 2, and 3 depict a preferred embodiment of the rehabilitation walking boot of the present invention. The embodiments are provided for descriptive purposes only. The patent application shall not be confined to a particular structure.

The rehabilitation walking boot comprises a foot support 10 and two leg supports 20. The foot support 10 is made up of a top surface 11, a bottom surface 12 and two lateral edges 13. The two lateral edges 13 are configured with slots 14 for insertion and fixation of the inserts 21 of the two leg supports.

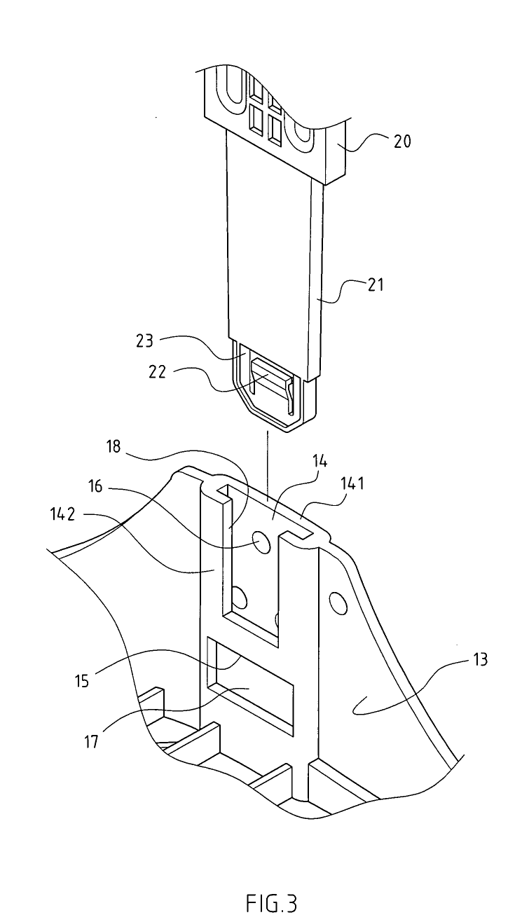

Said leg support 20 can be made of plastic, and its inserts 21 are configured with flexible positioning lockers 22 protruded on one side. Correspondingly on one side of the slots 14 of the lateral edges 13 of the foot support 10, a locking part 15 is configured. Moreover, the side wall of said slots 14 is configured with several perforated fixing holes 16. The inserts 21 of said leg support 20 can be configured with a ∩-shaped penetrated part 23 (see FIG. 3) to relatively form the flexible positioning locker 22. On one side of the slots 14 of the lateral edges 13 of the foot support 10, a perforation 17 can be configured so as to form said locking part 15 with the top edge of the perforation 17.

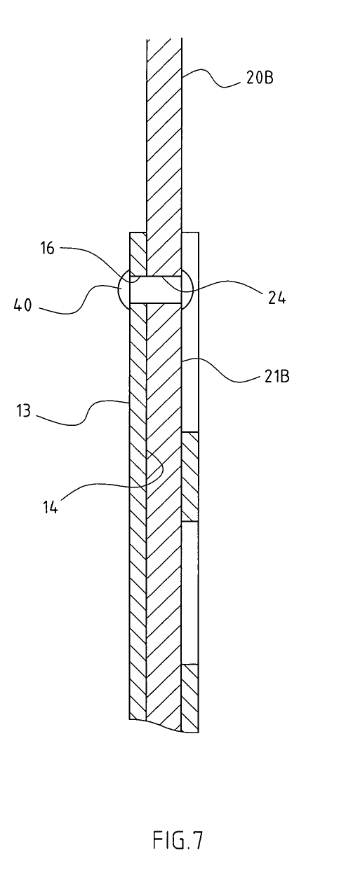

Said perforated fixing holes 16 can be configured on the outer side wall 141 of the slots 14. In the corresponding area of the inner side wall 142 of the slots 14, a cross-perforated concave 18 can be configured. Configuration of the perforated fixing holes 16 (see FIGS. 7 and 8) is for the purpose of fixing by the rivets 40 or bolts. Thus, when the slots 14 of the two lateral edges 13 of the foot support 10 are inserted into metal leg support 20b, the rivet 40 (or bolt) can go through the perforations 24 of the leg support 20b and the fixing holes 16 to fix the foot support and leg supports together.

The top surface 11 of the foot support 10 can be configured with multiple partitioned concave spaces 113 and partitions 115 in-between the concave spaces 113.

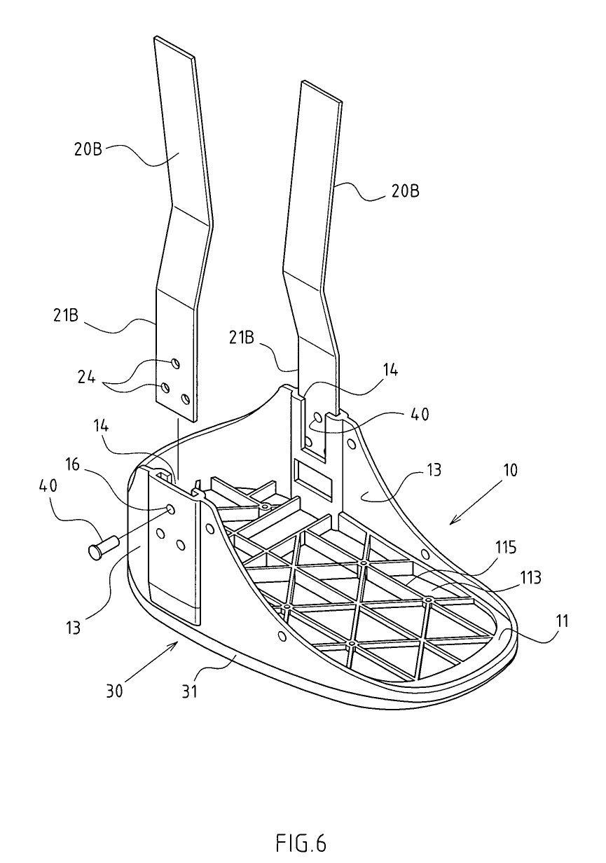

Referring to FIG. 6, the bottom surface 12 of the foot support 10 can be configured with a protrusion 121, and underneath the protrusion 121, a sole cover 30 can be fitted. On the periphery of the sole cover 30 is a collar 31 that extends from the sole cover vertically to tightly attach to the above-mentioned protrusion 121 of the bottom surface 12 of the foot support 10 (an adhesive can be used for fixation).

Referring to FIGS. 1, 2, and 6, the bottom surface 12 and said sole cover 30 of the foot support 10 can be shaped as a dome, and thus the sole cover 30 has a landing part 32, a front upswept part 33, and a rear upswept part 34. Referring to FIG. 6, between the front upswept part 33 and the landing part 32, there are at least 7 landing sections with different included angles with the horizon (1). The included angles between the 7 landing sections and the horizon are, in order, 5°, 6°, 8°, 9°, 10°, 12°, 14°. With these varied included angles, when the landing surface of the sole cover 30 touches the ground, an optimum leg movement reaction can be achieved. And consequently, when walking, the users can move their legs more smoothly, and the possible oppression on the wound can be minimized.

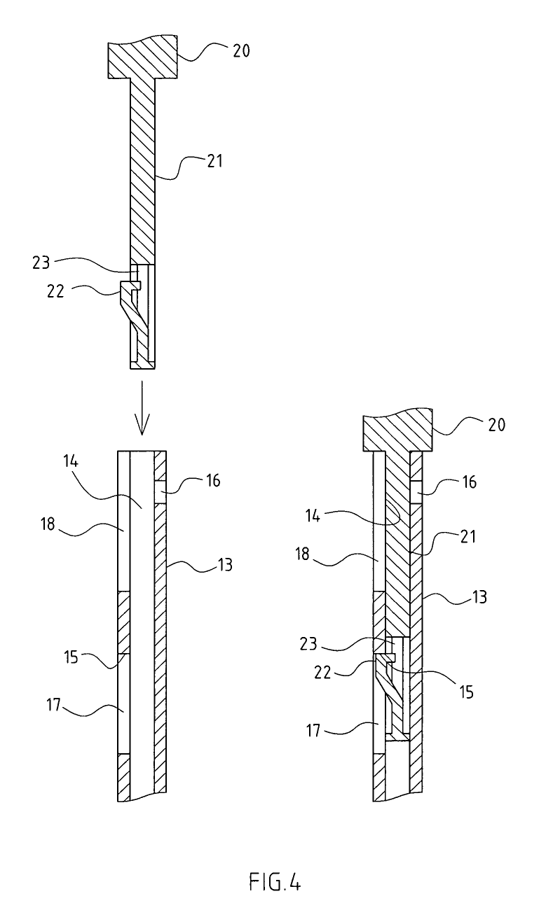

As described above, the present invention features a structure in which the inserts 21 of the leg support 20 are configured with a flexible positioning locker 22 that protrudes to one side. The lateral wall of the slots 14 of the foot support 10 lateral edges 13 is configured with perforated fixing holes 16, and makes it possible for the leg support of the rehabilitation walking boot to link to the foot support in either combined type or fixed type. In the combined type, as shown in FIG. 4, when the inserts 21 of the leg support 20 inserts into the slots 14 on the lateral edges of the foot support 10, a fixation can be achieved through the flexible positioning locker 22 protruding to one side locking the locking part 15. On the other hand, when the leg support 20 needs to be dismantled, the flexible positioning locker 22 is pressed inward, concurrently, the leg support 20 is pulled. Hence, in practice, it is more convenient and efficient than the prior art.

If the leg support uses the fixed type, as shown in FIGS. 7 and 8, when the inserts 21b of the leg support 20b are inserted into the slots 14 on the lateral edges 13 of the foot support 10, the leg support and the foot support are fixed together using rivets 40 (or bolts) to go through the perforations 24 of the leg support 20b and the perforated fixing holes 16 on the slots 14 of the foot support 10.

1. A of rehabilitation walking boot structure comprising:
a foot support, having a top surface, a bottom surface and two lateral edges, said two lateral edges being configured with slots;
two leg supports, each leg support having an insert to engage said slots on said two lateral edges of said foot support;
a flexible positioning locker protruded on one side, the locker being configured on an insert of one of the leg supports;
a locking part, being configured on one side of said slots on the lateral edges of said foot support; and correspondingly locking onto said flexible positioning locker; and
perforated fixing holes, being configured on side walls of the slots on the lateral edges of said foot support.

2. The boot structure defined in claim 1, wherein said insert of each leg support has a ∩-shaped penetrated part forming said flexible positioning locker; the slots of the lateral edges of said foot support, having one side with a perforation forming said locking part with a top edge of said perforation.

3. The boot structure defined in claim 1, wherein said perforated fixing holes are configured on an outer side wall of the slots, further comprising:
a cross-perforated concave in a corresponding area of an inner side wall of the slots, said perforated fixing holes being fixed by rivets or bolts.

4. The boot structure defined in claim 1, wherein said foot support has a top surface configured with a multiple partitioned concave spaces and partitions in-between the concave spaces.

5. The boot structure defined in claim 1, wherein said foot support has a bottom surface with a protrusion, said protrusion, having an underside with a sole cover fitted; thereon, said sole cover having a periphery with a collar extending therefrom vertically to tightly attach to said protrusion.

6. The boot structure defined in claim 5, wherein said bottom surface and said sole cover of said foot support has a shape of a dome, said sole cover having a landing part, a front upswept part, a rear upswept part, and seven landing sections, said seven landing sections being located, between said front upswept part and said landing part, said seven landing sections with different included angles with the horizon, said included angles being 5°, 6°, 8°, 9°, 10°, 12°, and 14°.
